(19) United States
(12) Patent Application Publication
(10) Pub. No.: US 2016/0067162 A1
(43) Pub. Date: Mar. 10, 2016

(54) PHOTOPROTECTIVE COMPOUNDS, COMPOSITIONS INCLUDING SAME AND USES THEREOF

(71) Applicants: UNIVERSITE DE RENNES 1, Rennes (FR); CENTRE NATIONAL DE LA RECHERCHE SCIENTIFIQUE-CNRS-, Paris Cedex 16 (FR)

(72) Inventors: Nicolas GOUAULT, Betton (FR); Khanh Hung NGUYEN, Ho Chi Minh (VN); Sophie TOMASI, Rennes (FR); Karine COSTUAS, Saint Aubin D'aubigne (FR)

(21) Appl. No.: 14/782,925

(22) PCT Filed: Apr. 4, 2014
(86) PCT No.: PCT/FR2014/050815

§ 371 Date: Oct. 7, 2015

(30) Foreign Application Priority Data
Apr. 8, 2013 (FR) 13 53106

(51) Int. Cl. A61K 8/49; A61Q 17/04; C07D 211/86

(52) U.S. Cl. CPC A61K 8/4926; C07D 211/86; A61Q 17/04

(57) ABSTRACT

A compound of formula (I):

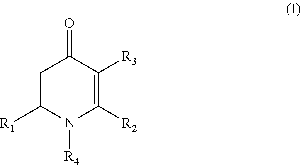

wherein:
R1 is H, benzyl, —CH₂—O-benzyl, a straight or branched C1-C12 alkyl radical, a hydroxyalkyl radical or a polyhydroxyalkyl radical,
R2 is H; a straight or branched C1-C12 alkyl radical; a —(CH₂)₃—CH═CH₂ radical; a phenyl, naphthyl, furan, thiophene or pyrazole radical, optionally substituted by at least one group selected among methoxy, phenoxy, halogen, dimethylamine, trifluoromethyl and alkynyl,
R3 is H; a vinyl radical optionally substituted by a carboxyalkyl; a nitrile; a carboxamide; a phenyl, naphthyl, furan, thiophene or pyrazole radical, optionally substituted by at least one group selected among methoxy, phenoxy, halogen, dimethylamine, trifluoromethyl and alkynyl; an aminoalkyl or aminocarboxyl radical; or an alkoxy radical,
R4 is H, a protective grouping, a straight or branched C1-C12 alkyl radical, a hydroxyalkyl radical or a polyhydroxyalkyl radical,
   and when:
R2 is H, R3 is —NHCOOC(CH₃)₃,
R2 is a propyl, R3 is —CH═CH—COO—C₂H₅ or R1=R3=R4=H,
R2 is a non-substituted phenyl, R3 is —CH═CH—COO—C₂H₅ or —NHCOOC(CH₃)₃,
R2 is a phenyl substituted in the para position, R3 is H.

The present invention relates to photoprotective compounds of formula (I), which have in particular a very good UVA-absorption capacity. The invention also relates to compositions comprising at least one of these compounds, and also to the use of these compounds as UV-screening agents.

It is known that UVA rays, having wavelengths of between 320 and 400 nm, cause browning of the skin, and are capable of inducing modification thereof, in particular in the case of sensitive skin or skin continually exposed to solar radiation. UVA rays cause in particular a loss of elasticity of the skin and the appearance of wrinkles resulting in premature skin aging. Thus, for esthetic and cosmetic reasons, such as conservation of the natural elasticity of the skin for example, an increasing number of people wish to control the effect of UVA rays on their skin. It is therefore desirable to screen out UVA radiation.

It is also known that light radiation having wavelengths of between 280 nm and 400 nm enables browning of human epidermis, and that rays having wavelengths more particularly between 280 and 320 nm, known as UVB, are harmful to the development of natural tanning. For these reasons and also for esthetic reasons, there is a constant demand for means for controlling this natural tanning with a view to thus controlling the color of the skin; it is therefore advisable to screen out this UVB radiation.

For the purpose of protecting the skin and keratin materials against UV radiation, antisun compositions comprising organic screening agents, which are active in the UVA range and active in the UVB range, are generally used. The majority of these screening agents are lipophilic, although they also group together hydrophilic compounds.

The development of cosmetic compositions which make it possible to effectively combat premature skin aging therefore involves the use of sunscreens, particularly those which absorb in the UVA range. These sunscreens make it possible to reduce the excess of photoinduced free radicals. However, with most compositions containing UV-screening agents, and in particular UVA-screening agents, the protection is not total. Thus, during repeated exposures, the residual amount of free radicals that persist despite protection by the UVA-screening agent can in the long term cause photoactinic aging phenomena.

One solution may consist in increasing the amounts of UVA-screening agents, but it is inadvisable to use excessively high amounts of screening agents in cosmetic products for daily care. This is because, with most screening agents, a maximum protective index is often reached, that is very difficult to improve by increasing the proportion of the screening agents.

Thus, the search for alternative solutions for protecting the skin against sun rays and for effectively combating premature skin aging under conditions of use and concentration that are acceptable by those skilled in the art remains topical.

There remains therefore the need to identify novel effective photoprotective compounds, which would in particular have a good UVA-absorption capacity and which would protect the skin against damage caused by sun rays.

The applicant has now just discovered, surprisingly, that it is possible to improve the protection of the skin and/or of the lips and/or of the hair against UV radiation, in particular with respect to UVA radiation, by using at least one compound of formula (I). These compounds in fact have a considerable UVA-absorption capacity and are very well tolerated.

A subject of the invention is therefore a compound of formula (I):

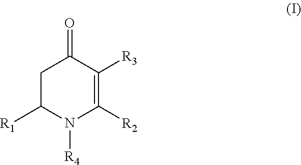

wherein:
R1 is H, benzyl, —CH₂—O-benzyl, a linear or branched C₁-C₁₂ alkyl radical, a hydroxyalkyl radical or a polyhydroxyalkyl radical,
R2 is H; a linear or branched C₁-C₁₂ alkyl radical; a —(CH₂)₃—CH═CH₂ radical; a phenyl, naphthyl, furan, thiophene or pyrazole radical, optionally substituted by at least one group selected from methoxy, phenoxy, halogen, dimethylamine, trifluoromethyl and alkynyl,
R3 is H; a vinyl radical optionally substituted by a carboxyalkyl; a nitrile; a carboxamide; a phenyl, naphthyl, furan, thiophene or pyrazole radical, optionally substituted by at least one group selected from methoxy, phenoxy, halogen, dimethylamine, trifluoromethyl and alkynyl; an aminoalkyl or aminocarboxyl radical; or an alkoxy radical,
R4 is H, a protective group, a linear or branched C₁-C₁₂ alkyl radical, a hydroxyalkyl radical or a polyhydroxyalkyl radical,
it being understood that:
when R2 is H, then R3 is —NHCOOC(CH₃)₃,
when R2 is a propyl, then R3 is —CH═CH—COO—C₂H₅ or R1=R3=R4=H,
when R2 is an unsubstituted phenyl, then R3 is selected from —CH═CH—COO—C₂H₅ and —NHCOOC(CH₃)₃, and
when R2 is a phenyl substituted in the para position, then R3 is H.

Preferably, when R1 is other than H, the starting amino acid (L or D) makes it possible to control the configuration of the final compound of formula (I).

In formula (I), the phenyl, naphthyl, furan, thiophene and pyrazole radicals can be substituted in various positions, such as in the ortho, meta or para position.

In the present invention, unless otherwise mentioned, the term “linear or branched C₁ to C₁₂ alkyl radical” is intended to mean the methyl, ethyl, isopropyl, n-propyl, n-butyl, i-butyl or t-butyl radical, the —C₅H₁₁ (pentyl) radical or the —C₉H₁₉ (nonyl) radical. Preferably, the linear or branched C₁ to C₁₂ alkyl radical is selected from the n-propyl, n-butyl, i-butyl and t-butyl radical.

In the present invention, unless otherwise mentioned, the term “hydroxyalkyl radical” is intended to mean a linear or branched C₁ to C₁₂ alkyl radical, substituted in the terminal position by an —OH. Likewise, the term “polyhydroxyalkyl radical” is intended to mean a linear or branched C₁ to C₁₂ alkyl radical, substituted at at least two carbon atoms by an —OH.

In the present invention, unless otherwise mentioned, the term “vinyl radical optionally substituted by a carboxyalkyl” is intended to mean a vinyl optionally substituted by a —COO—R group where R is a linear or branched C₁ to C₁₂ alkyl. Preferably, the vinyl radical optionally substituted by a carboxyalkyl is the —CH═CH—COO—C₂H₅ radical.

In the present invention, unless otherwise mentioned, the term “aminocarboxyl radical” is intended to mean an amino group substituted by a —COO—R group where R is a linear or branched C₁ to C₁₂ alkyl. Preferably, the aminocarboxyl radical is the —NHCOOC(CH₃)₃ radical.

Preferably, the alkoxy radical is selected from methoxy, ethoxy and propoxy. Preferably, it is the methoxy radical.

Preferably, the protective group is a group of the carbamate family; more preferentially, it is selected from the tert-butoxycarbonyl, carbobenzyloxy and 9-fluorenylmethyloxycarbonyl groups.

Preferably, the halogen is selected from iodine, bromine, chlorine and fluorine.

Preferably, the phenyl optionally substituted by at least one group selected from methoxy, phenoxy, halogen and dimethylamine, that can be used as the R2 radical, is selected from the unsubstituted phenyl, 4-chlorophenyl, 4-fluorophenyl, 2-methoxyphenyl, 4-methoxyphenyl, 4-dimethylaminophenyl, 3,5-dimethoxyphenyl and 4-phenoxyphenyl radicals.

Preferably, the naphthyl optionally substituted by at least one methoxy group, that can be used as the R2 radical, is selected from the unsubstituted naphthyl and 3-methoxynaphthyl radicals.

Preferably, the R2 radical is selected from a propyl radical, a —(CH₂)₃—CH═CH₂ radical, an unsubstituted phenyl, 4-chlorophenyl, 4-fluorophenyl, 2-methoxyphenyl, 4-methoxyphenyl, 4-dimethylaminophenyl, 3,5-dimethoxyphenyl, 3-methoxynaphthyl and 4-phenoxyphenyl.

Preferably, a subject of the invention is a compound of formula (I):

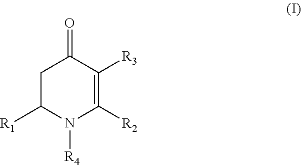

wherein:
R1 is H; benzyl or —CH₂—O-benzyl,
R2 is H; a linear or branched C₁-C₁₂ alkyl radical; a —(CH₂)₃—CH═CH₂ radical; a phenyl or naphthyl radical, optionally substituted by at least one group selected from methoxy, phenoxy, halogen and dimethylamine,
R3 is H; a vinyl radical optionally substituted by a carboxyalkyl; an aminocarboxyl radical; or a phenyl radical optionally substituted by at least one group selected from methoxy, phenoxy, halogen and dimethylamine,
R4 is H; a protective group or a linear or branched C₁-C₁₂ alkyl radical, it being understood that:
when R2 is H, then R3 is —NHCOOC(CH₃)₃,
when R2 is a propyl, then R3 is —CH═CH—COO—C₂H₅ or R1=R3=R4=H,
when R2 is an unsubstituted phenyl, then R3 is selected from —CH═CH—COO—C₂H₅ and —NHCOOC(CH₃)₃, and
when R2 is a phenyl substituted in the para position, then R3 is H.

Preferably, a subject of the invention is a compound of formula (I):

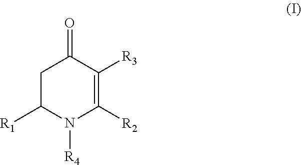

wherein:
R1 is H, benzyl or —CH₂—O-benzyl,
R2 is H; a propyl radical; a phenyl or naphthyl radical, optionally substituted by at least one group selected from methoxy, phenoxy, halogen and dimethylamine,
R3 is H; a vinyl radical optionally substituted by a carboxyalkyl; a phenyl radical optionally substituted by at least one group selected from methoxy, phenoxy, halogen and dimethylamine, or an aminocarboxyl radical,
R4 is H, a protective group or a linear or branched C₁-C₁₂ alkyl radical,
it being understood that:
when R2 is H, then R3 is —NHCOOC(CH₃)₃,
when R2 is a propyl, then R3 is —CH═CH—COO—C₂H₅ or R1=R3=R4=H,
when R2 is an unsubstituted phenyl, then R3 is selected from —CH═CH—COO—C₂H₅ and —NHCOOC(CH₃)₃, and
when R2 is a phenyl substituted in the para position, then R3 is H.

The compound of formula (I) according to the invention is preferably selected from:
6-(4-chlorophenyl)-2,3-dihydropyridin-4(1H)-one,
6-(4-fluorophenyl)-2,3-dihydropyridin-4(1H)-one,
6-(4-methoxyphenyl)-2,3-dihydropyridin-4(1H)-one,
6-(4-(dimethylamino)phenyl)-2,3-dihydropyridin-4(1H)-one,
6-propyl-2,3-dihydropyridin-4(1H)-one,
tert-butyl 5-(tert-butoxycarbonylamino)-4-oxo-6-phenyl-3,4-dihydropyridine-1(2H)-carboxylate,
(E)-tert-butyl 5-(3-ethoxy-3-oxoprop-1-enyl)-4-oxo-6-phenyl-3,4-dihydropyridine-1(2H)-carboxylate,
(E)-ethyl 3-(4-oxo-2-phenyl-1,4,5,6-tetrahydropyridin-3-yl)acrylate,
(R,E)-ethyl 3-(6-(benzyloxymethyl)-4-oxo-2-phenyl-1,4,5,6-tetrahydropyridin-3-yl)acrylate,
tert-butyl 6-(3,5-dimethoxyphenyl)-4-oxo-3,4-dihydropyridine-1(2H)-carboxylate,
tert-butyl 6-(6-methoxynaphthalen-2-yl)-4-oxo-3,4-dihydropyridine-1(2H)-carboxylate,
tert-butyl 4-oxo-6-(4-phenoxyphenyl)-3,4-dihydropyridine-1(2H)-carboxylate,
6-(3,5-dimethoxyphenyl)-2,3-dihydropyridin-4(1H)-one,
6-(4-phenoxyphenyl)-2,3-dihydropyridin-4(1H)-one,
6-(6-methoxynaphthalen-2-yl)-2,3-dihydropyridin-4(1H)-one,
6-(2-methoxyphenyl)-2,3-dihydropyridin-4(1H)-one,
(S,E)-ethyl 3-(6-benzyl-4-oxo-2-propyl-1,4,5,6-tetrahydropyridin-3-yl)acrylate, and
tert-butyl 1-methyl-4-oxo-1,4,5,6-tetrahydropyridin-3-ylcarbamate.

The compounds of formula (I) according to the invention can in particular be synthesized according to the following scheme:

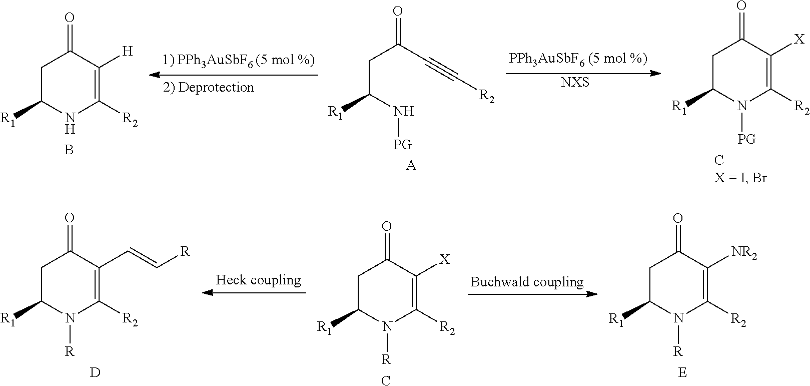

The compounds B, for which R³=H, are readily accessible from the intermediate A by cyclization catalyzed by gold(I) complexes then deprotection of the nitrogen in order to obtain the compounds of formula (I) wherein R⁴ represents a hydrogen atom. The compounds C—halogenated pyridines—are obtained by cyclization by catalysis with gold in the presence of N-halosuccinimide (NXS). These intermediates C can then be functionalized by palladium-catalyzed reactions, of Suzuki-Miyaura, Heck or Buchwald coupling. These palladium-catalyzed coupling reactions make it possible to introduce various groups at the position R³. R³ is an alkenyl group in the case of Heck couplings, an aryl group in the case of Suzuki-Miyaura couplings and an aminoalkyl or aminoaryl or aminocarboxyalkyl group in the case of Buchwald-type couplings.

A subject of the present invention is also a composition comprising, in a cosmetically acceptable medium, at least one compound of formula (I) according to the invention. This composition is called “composition according to the invention” in the present application.

The term “cosmetically acceptable” is intended to mean compatible with the skin and/or skin appendages, which has a pleasant color, odor and feel and which does not generate unacceptable discomfort (tingling, tautness, redness) capable of dissuading the consumer from using this composition.

The composition according to the invention may comprise a compound of formula (I), two compounds of formula (I), or more than two compounds of formula (I).

Preferably, the compound(s) of formula (I) is (are) present in the composition according to the invention in proportions ranging from 0.01% to 20% by weight relative to the total weight of the composition, and preferably ranging from 0.1% to 10% by weight relative to the total weight of the composition.

The composition according to the invention may also comprise at least one additional compound which screens out UVA and/or UVB radiation. This additional compound which screens out UVA and/or UVB radiation is distinct from the compounds of formula (I).

The expression “compound which screens out UV (UVA and/or UVB) radiation” is intended to mean a substance capable of absorbing at least one part of the UV (UVA and/or UVB) radiation emitted by the sun in order to protect the skin and/or the lips and/or the hair against the harmful effects of this radiation. The wavelengths of UVA radiation are different than the wavelengths of UVB radiation. The wavelengths of UVA radiation are between 400 nm and 315 nm; those of UVB radiation are between 315 nm and 280 nm.

The additional compound which screens out UVA and/or UVB radiation can be selected from organic or inorganic screening agents active in the UVA and/or UVB range, which are hydrophilic or lipophilic or else insoluble in the cosmetic solvents commonly used.

The term “lipophilic UV-screening agent” is intended to mean any cosmetic or dermatological screening agent capable of being completely dissolved in the molecular state in a liquid fatty phase or else of being solubilized in colloidal form (for example in micellar form) in a liquid fatty phase.

The term “hydrophilic UV-screening agent” is intended to mean any cosmetic or dermatological screening agent capable of being completely dissolved in the molecular state in a liquid aqueous phase or else of being solubilized in colloidal form (for example in micellar form) in a liquid aqueous phase.

The term “insoluble UV-screening agent” is intended to mean any cosmetic or dermatological screening agent which is defined neither as a lipophilic UV-screening agent nor as a hydrophilic UV-screening agent, and which is in the form of particles in an aqueous or fatty liquid phase.

The additional compound which screens out UVA and/or UVB radiation is in particular selected from anthranilics; cinnamic derivatives; salicylic derivatives; benzophenone derivatives; phenyl benzotriazole derivatives; benzalmalonate derivatives, in particular those mentioned in patent U.S. Pat. No. 5,624,663; phenyl benzimidazole derivatives; imidazolines; 4,4-diarylbutadiene derivatives; bisbenzoazolyl derivatives as described in patents EP 669323 and U.S. Pat. No. 2,463,264; p-aminobenzoic acid (PABA) derivatives; methylene bis(hydroxyphenyl benzotriazole) derivatives as described in applications U.S. Pat. No. 5,237,071, U.S. Pat. No. 5,166,355, GB 2303549, DE 197 26 184 and EP 893119; benzoxazole derivatives as described in patent applications EP 0832642, EP 1027883, EP 1300137 and DE 10162844; screening polymers and screening silicones such as those described in particular in application WO 93/04665; a-alkylstyrene-based dimers such as those described in patent application DE 19855649; 4,4-diarylbutadienes as described in applications EP 0967200, DE 19746654, DE 19755649, EP-A-1008586, EP 1133980 and EP 133981; merocyanine derivatives such as those described in applications WO 04006878, WO 05058269 and WO 06032741; and mixtures thereof.

As examples of organic additional compounds which screen out UVA and/or UVB radiation, mention may be made of those denoted below under their INCI name, and classified according to the UVA and/or UVB radiation wavelength range:

### I/Lipophilic UVA-Screening Agents

Dibenzoylmethane derivatives:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-tert-butyl-4′-methoxydibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane,
2,6-dimethyl-4-tert-butyl-4′-methoxydibenzoylmethane.

Among the dibenzoylmethane derivatives mentioned above, use is, for example, made of 4-isopropyldibenzoylmethane, sold under the name Eusolex 8020 by the company Merck, or 4-tert-butyl-4′-methoxydibenzoylmethane or butyl methoxy dibenzoylmethane or avobenzone, proposed for sale under the trade name Parsol 1789 by the company DSM Nutritional Products, Inc.

### Aminobenzophenones:

n-Hexyl 2-(4-diethylamino-2-hydroxybenzoyl)benzoate sold in particular under the trade name Uvinul A+ by BASF; 1,1′-(1,4-piperazinediyl)bis[1-[2-[4-(diethylamino)-2-hydroxybenzoyl]phenyl]methanone] (CAS 919803-06-8);

### Anthranilic Derivatives:

Menthyl anthranilate sold in particular under the trade name Neo Heliopan MA by Symrise;

### 4,4-Diarylbutadiene Derivatives:

1,1-Dicarboxy(2,2′-dimethylpropyl)-4,4-diphenylbutadiene;

### Merocyanine Derivatives:

Octyl-5-N,N-diethylamino-2-phenylsulfonyl-2,4-pentadienoate.

### II/Hydrophilic UVA-Screening Agents

Bisbenzoazolyl derivatives as described in patents EP 669 323 and U.S. Pat. No. 2,463,264 and more particularly the compound disodium phenyl dibenzimidazo tetrasulfonate sold under the trade name Neo Heliopan AP by Symrise.

The preferred hydrophilic UVA-screening agent is terephthalylidene dicamphor sulfonic acid, sold under the name Mexoryl SX by Chimex.

### III/Lipophilic UVB-Screening Agents

### Para-Aminobenzoates:

Ethyl PABA; ethyl dihydroxypropyl PABA; ethylhexyl dimethyl PABA (Escalol 507 from ISP);

### Salicylic Derivatives:

Homosalate sold in particular under the name Eusolex HMS by Rona/EM Industries; ethylhexyl salicylate sold in particular under the name Neo Heliopan OS by Symrise; dipropylene glycol salicylate sold in particular under the name Dipsal by Scher; TEA salicylate under the name Neo Heliopan TS by Symrise;

### Cinnamates:

Ethylhexyl methoxycinnamate sold in particular under the trade name Parsol MCX by DSM Nutritional Products, Inc.; isopropyl methoxy cinnamate; isoamyl methoxy cinnamate sold in particular under the trade name Neo Heliopan E 1000 by Symrise; diisopropyl methylcinnamate; cinnoxate; glyceryl ethylhexanoate dimethoxycinnamate; etocrylene, sold in particular under the trade name Uvinul N35 by BASF; octocrylene, sold in particular under the trade name Uvinul N539 by BASF;

### Benzylidenecamphor Derivatives:

3-Benzylidenecamphor produced under the name Mexoryl SD by Chimex; methylbenzylidenecamphor sold in particular under the name Eusolex 6300 by Merck; polyacrylamidomethylbenzylidenecamphor produced under the name Mexoryl SW by Chimex;

### Triazine Derivatives:

Ethylhexyl triazone sold in particular under the trade name Uvinul T150 by BASF; diethylhexyl butamido triazone sold in particular under the trade name Uvasorb HEB by Sigma 3V; 2,4,6-tris(dineopentyl 4′-aminobenzalmalonate)-s-triazine; 2,4,6-tris(diisobutyl 4′-amino-benzalmalonate)-s-triazine; 2,4-bis(dineopentyl 4′-aminobenzalmalonate)-6-(n-butyl 4′-aminobenzoate)-s-triazine; 2,4-bis(n-butyl 4′-aminobenzoate)-6-(aminopropyltrisiloxane)-s-triazine; the symmetrical triazine screening agents described in patent U.S. Pat. No. 6,225,467, application WO 2004/085412 (see compounds 6 and 9) or the document “Symmetrical Triazine Derivatives” IP. COM Journal, IP. COM INC West Henrietta, N.Y., US (Sep. 20, 2004), in particular 2,4,6-tris(biphenyl)-1,3,5-triazine (in particular 2,4,6-tris(biphenyl-4-yl-1,3,5-triazine) and 2,4,6-tris(terphenyl)-1,3,5-triazine);

### Imidazoline Derivatives:

Ethylhexyl dimethoxybenzylidene dioxoimidazoline propionate;

### Benzalmalonate Derivatives:

Polyorganosiloxanes comprising a benzalmalonate function, such as the Polysilicone-15 sold in particular under the trade name Parsol SLX by DSM Nutritional Products, Inc.; dineopentyl 4′-methoxybenzalmalonate.

### IV/Hydrophilic UVB-Screening Agents

The Following p-Aminobenzoic Acid (PABA) Derivatives:

PABA, glyceryl PABA and PEG-25 PABA sold in particular under the trade name Uvinul P25 by BASF;

The phenylbenzimidiazole sulfonic acid sold in particular under the trade name Eusolex 232 by Merck, ferulic acid, salicylic acid, DEA methoxycinnamate, the benzylidenecamphor sulfonic acid produced under the name Mexoryl SL by Chimex, the camphor benzalkonium methosulfate produced under the name Mexoryl SO by Chimex.

### V/Lipophilic Mixed UVA- and UVB-Screening Agents

### Benzophenone Derivatives:

Benzophenone-1 sold in particular under the trade name Uvinul 400 by BASF; benzophenone-2 sold in particular under the trade name Uvinul D50 by BASF; benzophenone-3 or oxybenzone sold in particular under the trade name Uvinul M40 by BASF; benzophenone-6 sold in particular under the trade name Helisorb 11 by Norquay; benzophenone-8 sold in particular under the trade name Spectra-Sorb UV-24 by American Cyanamid; benzophenone-10; benzophenone-11 or benzophenone-12;

### Phenyl Benzotriazole Derivatives:

Drometrizole trisiloxane sold in particular under the name Silatrizole by Rhodia Chimie or produced under the name Meroxyl XL by the company Chimex; methylenebisbenzo-triazolyltetramethylbutylphenol, sold in solid form in particular under the trade name Mixxim BB/100 by Fairmount Chemical or in micronized form as an aqueous dispersion in particular under the trade name Tinosorb M by Ciba Specialty Chemicals;

### Benzoxazole Derivatives:

2,4-bis-[5-(1,1-dimethylpropyl)benzoxazol-2-yl-(4-phenyl)imino]-6-(2-ethylhexyl)imino-1,3,5-triazine sold in particular under the name Uvasorb K2A by Sigma 3V;

### Bis-Resorcinyltriazine Derivatives:

2,4-bis{[4-(2-ethylhexyloxy)-2-hydroxy]phenyl}-6-(4-methoxyphenyl)-1,3,5-triazine or bisethylhexyloxyphenol methoxyphenyl triazine (INCI name) such as the product sold under the trade name Tinosorb S by Ciba Geigy;
2,4-bis{[4-(3-(2-propyloxy)-2-hydroxypropyloxy)-2-hydroxy]phenyl}-6-(4-methoxyphenyl)-1,3,5-triazine;
2,4-bis{[4-(2-ethylhexyloxy)-2-hydroxy]phenyl}-6-[4-(2-methoxyethylcarboxy)phenylamino]-1,3,5-triazine;
2,4-bis{[4-tris(trimethylsiloxysilylpropyloxy)-2-hydroxyphenyl}-6-(4-methoxyphenyl)-1,3,5-triazine;
2,4-bis{[4-(2″-methylpropenyloxy)-2-hydroxy]phenyl}-6-(4-methoxyphenyl)-1,3,5-triazine;
2,4-bis{[4-(1′,1′,1′,3′,5′,5′,5′-heptamethyltrisiloxy-2″-methylpropyloxy)-2-hydroxy]phenyl}-6-(4-methoxyphenyl)-1,3,5-triazine;
2,4-bis{[4-(3-(2-propyloxy)-2-hydroxypropyloxy)-2-hydroxy]phenyl}-6-[(4-ethylcarboxy)phenylamino]-1,3,5-triazine;
2,4-bis{[4-(2-ethylhexyloxy)-2-hydroxy]phenyl}-6-(1-methylpyrrol-2-yl)-1,3,5-triazine.

### VI/Hydrophilic Mixed UVA- and UVB-Screening Agents

Benzophenone derivatives comprising at least one sulfonic radical, such as benzophenone-4 sold in particular under the trade name Uvinul MS 40 by BASF, benzophenone-5 and benzophenone-9.

The additional compound which screens out UVA and/or UVB radiation can also be selected from mineral screening agents, which are pigments. The pigments may be coated or uncoated. The coated pigments are pigments which have undergone one or more surface treatments of chemical, electronic, mechanochemical and/or mechanical nature with compounds as described, for example, in Cosmetics & Toiletries, February 1990, Vol. 105, p. 53-64, such as amino acids, beeswax, fatty acids, fatty alcohols, anionic surfactants, lecithins, sodium, potassium, zinc, iron or aluminum salts of fatty acids, metal alkoxides (of titanium or of aluminum), polyethylene, silicones, proteins (collagen, elastin), alkanolamines, silicon oxides, metal oxides or sodium hexametaphosphate. Of course, the metal oxide pigments, before treatment thereof with silicones, may have been treated with other surface agents, in particular with cerium oxide, alumina, silica, aluminum compounds, silicon compounds, or mixtures thereof. The coated pigments are, for example, titanium oxides coated:
with silica, such as the product Sunveil from the company Ikeda and the product Eusolex T-AVO from the company Merck,
with silica and with iron oxide, such as the product Sunveil F from the company Ikeda,
with silica and with alumina, such as the products Microtitanium Dioxide MT 500 SA and Microtitanium Dioxide MT 100 SA from the company Tayca, Tioveil from the company Tioxide, and Mirasun TiW 60 from the company Rhodia,
with alumina, such as the products Tipaque TTO-55 (B) and Tipaque TTO-55 (A) from the company Ishihara, and UVT 14/4 from the company Kemira,
with alumina and with aluminum stearate, such as the product Microtitanium Dioxide MT 100 TV, MT 100 TX, MT 100 Z or MT-01 from the company Tayca, the products Solaveil CT-10 W, Solaveil CT 100 and Solaveil CT 200 from the company Uniqema,
with silica, with alumina and with alginic acid, such as the product MT-100 AQ from the company Tayca,
with alumina and with aluminum laurate, such as the product Microtitanium Dioxide MT 100 S from the company Tayca,
with iron oxide and with iron stearate, such as the product Microtitanium Dioxide MT 100 F from the company Tayca,
with zinc oxide and with zinc stearate, such as the product BR351 from the company Tayca,
with silica and with alumina and treated with a silicone, such as the products Microtitanium Dioxide MT 600 SAS, Microtitanium Dioxide MT 500 SAS or Microtitanium Dioxide MT 100 SAS from the company Tayca,
with silica, with alumina and with aluminum stearate and treated with a silicone, such as the product STT-30-DS from the company Titan Kogyo,
with silica and treated with a silicone, such as the product UV-Titan X 195 from the company Kemira, or the product SMT-100 WRS from the company Tayca,
with alumina and treated with a silicone, such as the products Tipaque TTO-55 (S) from the company Ishihara, or UV Titan M 262 from the company Kemira, or with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca.

Other titanium oxide pigments treated with a silicone are, for example, TiO₂ treated with octyltrimethylsilane, such as the product sold under the trade name T 805 by the company Degussa Silices, TiO₂ treated with a polydimethylsiloxane, such as the product sold under the trade name 70250 Cardre UF TiO2S13 by the company Cardre, anatase/rutile TiO₂ treated with a polydimethylhydrogenosiloxane, such as the product sold under the trade name Micro Titanium Dioxyde USP Grade Hydrophobic by the company Color Techniques.

The uncoated titanium oxide pigments are, for example, sold by the company Tayca under the trade names Microtitanium Dioxide MT 500 B or Microtitanium Dioxide MT 600 B, or by the company Degussa under the name P 25, by the company Wackher under the name Transparent Titanium Oxide PW, by the company Miyoshi Kasei under the name UFTR, by the company Tomen under the name ITS and by the company Tioxide under the name Tioveil AQ.

The uncoated zinc oxide pigments are for example those sold under the name Z-cote by the company Sunsmart, or those sold under the name Nanox by the company Elementis.

The coated zinc oxide pigments are, for example, those sold under the name Z-cote HP1 by the company Sunsmart (dimethicone-coated ZnO); those sold under the name Zinc Oxide CS-5 by the company Toshibi (ZnO coated with polymethylhydrogenosiloxane); those sold under the name Nanogard Zinc Oxide FN by the company Nanophase Technologies (as a dispersion at 40% in Finsolv TN, C₁₂-C₁₅ alkyl benzoate); those sold under the name Daitopersion Zn-30 and Daitopersion Zn-50 by the company Daito (dispersions in cyclopolymethylsiloxane/oxyethylenated polydimethylsiloxane, containing 30% or 50% of zinc oxides coated with silica and polymethylhydrogenosiloxane); those sold under the name NFD Ultrafine ZnO by the company Daikin (ZnO coated with perfluoroalkyl phosphate and perfluoroalkylethyl-based copolymer in dispersion in cyclopentasiloxane); those sold under the name SPD-Z1 by the company Shin-Etsu (ZnO coated with silicone-grafted acrylic polymer, dispersed in cyclodimethylsiloxane); those sold under the name Escalol Z100 by the company ISP (ZnO treated with alumina and dispersed in the ethylhexyl methoxycinnamate/PVP-hexadecene copolymer/methicone mixture); those sold under the name Fuji ZnO-SMS-10 by the company Fuji Pigment (ZnO coated with silica and polymethylsilsesquioxane); or those sold under the name Nanox Gel TN by the company Elementis (ZnO dispersed at 55% in C₁₂-C₁₅ alkyl benzoate with hydroxystearic acid polycondensate).

The uncoated cerium oxide pigments are sold, for example, under the name Colloidal Cerium Oxide by the company Rhone Poulenc. The uncoated iron oxide pigments are, for example, sold by the company Arnaud under the names Nanogard WCD 2002 (FE 45B), Nanogard Iron FE 45 BL AQ, Nanogard FE 45R AQ and Nanogard WCD 2006 (FE 45R), or by the company Mitsubishi under the name TY-220. The coated iron oxide pigments are, for example, sold by the company Arnaud under the names Nanogard WCD 2008 (FE 45B FN), Nanogard WCD 2009 (FE 45B 556), Nanogard FE 45 BL 345 and Nanogard FE 45 BL, or by the company BASF under the name Transparent Iron Oxide.

Mention may also be made of the mixtures of metal oxides, in particular of titanium dioxide and of cerium dioxide, including the equal-weight mixture of silica-coated titanium dioxide and silica-coated cerium dioxide, sold by the company Ikeda under the name Sunveil A, and also the mixture of titanium dioxide and zinc dioxide coated with alumina, with silica and with silicone, such as the product M 261 sold by the company Kemira, or coated with alumina, with silica and with glycerol, such as the product M 211 sold by the company Kemira.

The composition of the invention preferably also comprises cosmetically acceptable excipients.

The composition may also comprise at least one additional ingredient intended to provide an immediate visual effect. Mention may in particular be made of soft-focus fillers or agents which promote the naturally pinkish coloration of the skin. As agents which promote the naturally pinkish coloration of the skin, mention may, for example, be made of self-tanning agents, i.e. an agent which, when applied to the skin, in particular to the face, makes it possible to obtain a tanning effect more or less similar in appearance to that which can result from prolonged exposure to the sun (natural tanning) or under a UV lamp. As examples of self-tanning agents, mention may in particular be made of: mono- or polycarbonylated compounds such as, for example, isatin, alloxan, ninhydrin, glyceraldehyde, meso-tartaric aldehyde, glutaraldehyde, erythrulose, pyrazoline-4,5-dione derivatives as described in patent applications FR 2 466 492 and WO 97/35842, dihydroxyacetone (DHA), 4,4-dihydroxypyrazoline-5-one derivatives as described in patent application EP 903 342.

Other dyes which make it possible to modify the color produced by the self-tanning agent may also be used. These dyes can be selected from synthetic or natural direct dyes selected, for example, from anthraquinones, caramel, carmine, carbon black, azulene blues, methoxalene, trioxalene, guajazulene, chamazulene, rose bengal, cosine 10B, cyanosine, daphinine, indole derivatives, for instance monohydroxyindoles, as described in patent FR 2 651 126 (i.e.: 4-, 5-, 6- or 7-hydroxyindole) or dihydroxyindoles as described in patent EP-B-0 425 324 (2,3-dimethyl-5,6-dihydroxyindole).

The compositions in accordance with the present invention may also comprise conventional cosmetic adjuvants in particular selected from fatty substances, in particular oils, waxes, organic solvents, ionic or nonionic thickeners, stabilizers, emollients, silicones, antifoams, fragrances, preservatives, anionic, cationic, nonionic, zwitterionic or amphoteric surfactants, active agents, fillers, polymers, propellants, basifying or acidifying agents or any other ingredient normally used in the cosmetics and/or dermatological field.

The fatty substances may consist of an oil or a wax or mixtures thereof.

The term “oil” is intended to mean a compound that is liquid at ambient temperature.

The term “wax” is intended to mean a compound that is solid or substantially solid at ambient temperature, and the melting point of which is generally above 35° C.

As oils, mention may, for example, be made of mineral oils (liquid paraffin); vegetable oils (sweet almond oil, macadamia oil, blackcurrant seed oil, jojoba oil); synthetic oils, for instance perhydrosqualene, fatty alcohols, fatty amides (for instance the isopropyl lauroyl sarcosinate sold under the name Eldew SL-205 by the company Ajinomoto), fatty acids or esters, for instance the C₁₂-C₁₅ alkyl benzoate sold under the trade name Finsolv TN or Witconol TN by the company Witco, 2-ethylphenyl benzoate, for instance the commercial product sold under the name X-Tend 226 by the company ISP, octyl palmitate, isopropyl lanolate, triglycerides, including those of capric/caprylic acid, the dicaprylyl carbonate sold under the name Cetiol CC by the company Cognis, oxyethylenated or oxypropylenated fatty esters and ethers; silicone oils (cyclomethicone, polydimethysiloxanes or PDMSs) or fluoro oils, polyalkylenes, trialkyl trimellitates, for instance tridecyl trimellitate.

As wax, mention may, for example, be made of carnauba wax, beeswax, hydrogenated castor oil, polyethylene waxes and polymethylene waxes, for instance the products sold under the name Cirebelle 303 by the company Sasol.

Among the organic solvents, mention may, for example, be made of lower alcohols and lower polyols. The latter may be selected from glycols and glycol ethers, for instance ethylene glycol, propylene glycol, butylene glycol, dipropylene glycol or diethylene glycol.

As hydrophilic thickeners, mention may, for example, be made of carboxyvinyl polymers such as carbopols (carbomers) and pemulens (acrylate/C₁₀-C₃₀ alkyl acrylate copolymer); polyacrylamides, for instance the crosslinked copolymers sold under the names Sepigel 305 (CTFA name: polyacrylamide/C13-14 isoparaffin/laureth 7) or Simulgel 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name Hostacerin AMPS (CTFA name: ammonium polyacryloyldimethyl taurate) or Simulgel 800 sold by the company SEPPIC (CTFA name: sodium polyacryloyldimethyl taurate/polysorbate 80/sorbitan oleate); copolymers of 2-acrylamido-2-methylpropanesulfonic acid and of hydroxyethyl acrylate, for instance Simulgel NS and Sepinov EMT 10 sold by the company SEPPIC; cellulose-based derivatives such as hydroxyethylcellulose; polysaccharides and in particular gums such as xanthan gum; hydrophilic or hydrodispersible silicone derivatives, for instance acrylic silicones, silicone polyethers and cationic silicones and mixtures thereof. As lipophilic thickeners, mention may, for example, be made of synthetic polymers such as the poly(C₁₀-C₃₀ alkyl acrylate)s sold under the name Intelimer IPA 13-1 and Intelimer IPA 13-6 by the company Landec or else modified clays such as hectorite and derivatives thereof, for instance the products sold under the Bentone names.

Of course, those skilled in the art will take care to select the optional additional compound(s) mentioned above and/or the amounts thereof in such a way that the advantageous properties intrinsically associated with the compositions in accordance with the invention are not, or not substantially, impaired by the envisioned addition(s), in particular the improvement of the protection of the skin and/or of the hair and/or of the lips against sun rays.

The compositions according to the invention can be prepared according to techniques well known to those skilled in the art. They can in particular be in the form of a simple or complex emulsion (O/W, W/O, O/W/O or W/O/W) such as a cream, a milk or a cream gel; in the form of an aqueous gel; or in the form of a lotion. They can optionally be packaged as an aerosol or be in the form of a foam or a spray. The compositions according to the invention can be in the form of an oil-in-water or water-in-oil emulsion.

Emulsions generally contain at least one emulsifying surfactant selected from amphoteric, anionic, cationic or nonionic emulsifying surfactants, used alone or as a mixture. The emulsifiers are selected appropriately according to the emulsion to be obtained (W/O or O/W). As emulsifying surfactants that can be used for preparing the W/O emulsions, mention may, for example, be made of alkyl esters or ethers of sorbitan, of glycerol or of sugars; silicone surfactants, for instance dimethicone copolyols, such as the mixture of cyclomethicone and of dimethicone copolyol, sold under the name DC 5225 C by the company Dow Corning, and alkyl dimethicone copolyols, such as the lauryl methicone copolyol sold under the name Dow Corning 5200 Formulation Aid by the company Dow Corning; cetyl dimethicone copolyol, such as the product sold under the name Abil EM 90R by the company Goldschmidt and the mixture of cetyl dimethicone copolyol, polyglyceryl (4 mol) isostearate and hexyl laurate sold under the name Abil WE 09 by the company Goldschmidt. One or more coemulsifiers, which advantageously can be selected from the group comprising alkyl esters of polyol, can also be added thereto.

As alkyl esters of polyol, mention may in particular be made of polyethylene glycol esters, for instance PEG-30 dipolyhydroxystearate, such as the product sold under the name Arlacel P135 by the company ICI.

As esters of glycerol and/or of sorbitan, mention may, for example, be made of polyglyceryl isostearate, such as the product sold under the name Isolan GI 34 by the name Arlacel 987 by the company ICI; glyceryl sorbitan isostearate, such as the product sold under the name Arlacel 986 by the company ICI, and mixtures thereof.

For the O/W emulsions, mention may be made, for example, as emulsifying surfactants, of nonionic emulsifiers, such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters, for instance the PEG-100 stearate/glyceryl stearate mixture sold, for example, by the company ICI under the name Arlacel 165; oxyalkylenated (oxyethylenated and/or oxypropylenated) fatty alcohol ethers; sugar esters, for instance sucrose stearate; ethers of fatty alcohol and of sugar, in particular alkylpolyglucosides (APGs), such as the decylglucoside and the laurylglucoside sold, for example, by the company Henkel under the respective names Plantaren 2000 and Plantaren 1200, the cetostearylglucoside optionally as a mixture with cetostearyl alcohol, sold for example under the name Montanov 68 by the company SEPPIC, under the name Tegocare CG90 by the company Goldschmidt and under the name Emulgade KE3302 by the company Henkel, and also arachidyl glucoside, for example in the form of the mixture of arachidyl and behenyl alcohols and of arachidylglucoside sold under the name Montanov 202 by the company SEPPIC. According to one particular embodiment of the invention, the mixture of the alkylpolyglucoside as defined above with the corresponding fatty alcohol may be in the form of a self-emulsifying composition, as described, for example, in document WO-A-92/06778.

The compounds of formula (I) and the compositions comprising them according to the invention have applications in a large number of treatments, in particular cosmetic treatments, for the skin, the lips and/or the hair, including the scalp, in particular for protecting and/or caring for the skin, the lips and/or the hair, and/or for making up the skin and/or the lips.

A subject of the present invention is therefore also the non-therapeutic cosmetic use of at least one compound of formula (I) according to the invention, for protecting the skin and/or the lips and/or the hair against solar radiation.

Finally, a subject of the present invention is a cosmetic process for protecting the skin and/or the lips and/or the hair, comprising the application of at least one compound of formula (I), or of a composition according to the invention, to the skin and/or the lips and/or the hair.

The compositions according to the invention can, for example, be used as a makeup product. The compositions according to the invention can also be used as a care and/or solar protection product for the face and/or the body, which is of liquid to semi-liquid consistency, such as milks, more or less smooth creams, cream gels, or pastes. They can optionally be packaged as an aerosol and be in the form of a foam or a spray.

Concrete, but in no way limiting, examples illustrating the invention will now be given. In the examples, unless otherwise indicated, the temperature is expressed in degrees Celsius and the pressure is atmospheric pressure. The amounts of the ingredients of the compositions are given as % by weight relative to the total weight of the composition.

The examples which follow refer to the appended figures, which describe the UV spectra obtained for compounds according to the invention.

In particular:

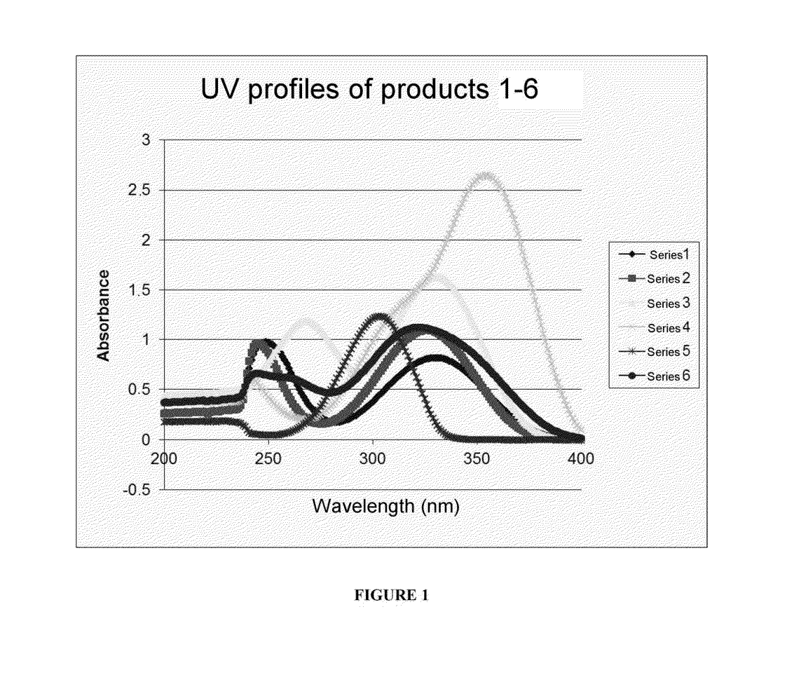

FIG. 1 presents the UV spectra obtained for compounds 1 to 6 of example 2 below:

Compound 1=Series 1

Compound 2=Series 2 . . . Compound 6=Series 6.

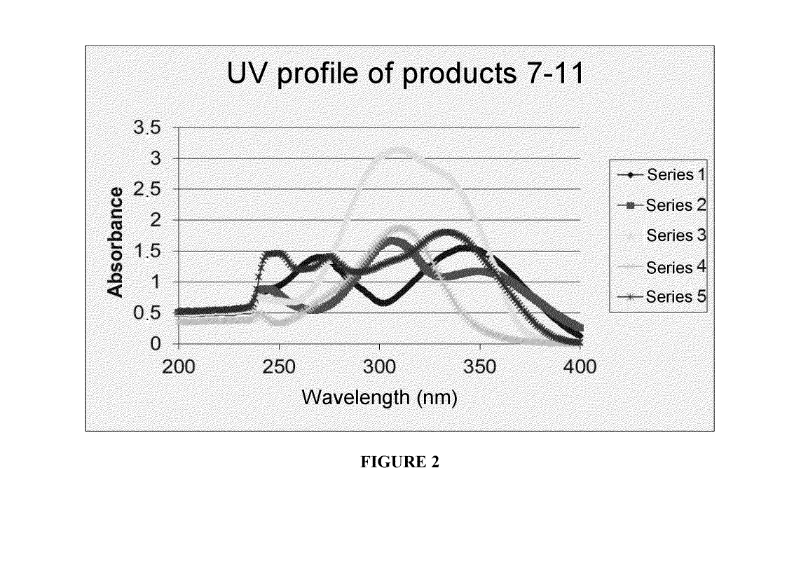

FIG. 2 presents the UV spectra obtained for compounds 7 to 11 of example 2 above:

Compound 7=Series 1

Compound 8=Series 2

Compound 9=Series 3

Compound 10=Series 4

Compound 11=Series 5.

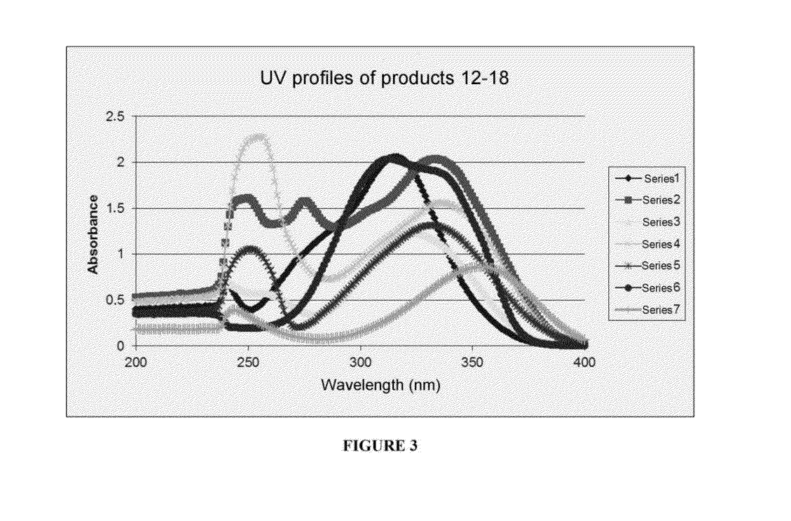

FIG. 3 presents the UV spectra obtained for compounds 12 to 18 of example 2 below:

Compound 12=Series 1

Compound 13=Series 2

Compound 14=Series 3

Compound 15=Series 4

Compound 16=Series 5

Compound 17=Series 6

Compound 18=Series 7.

### EXAMPLES

### Example 1

### Synthesis of the Compounds of Formula (I)

The general scheme is the following:

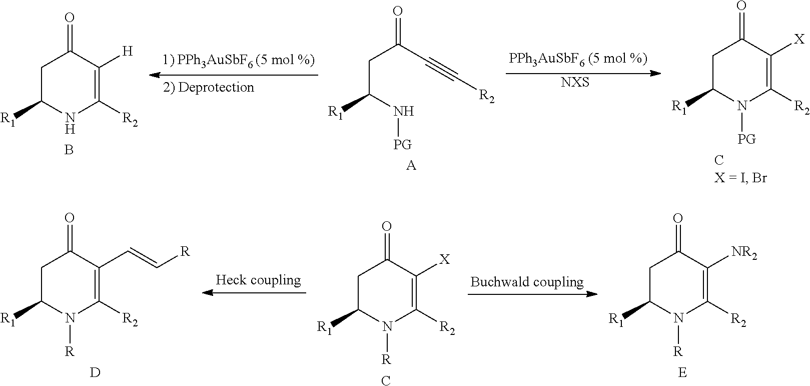

### General Experimental Methods:

All the high-quality reagents were purchased from commercial suppliers, and used without additional purification or were purified/dried according to Armarego WLF and Chai CLL (Purification de produits chimiques de laboratoire [Purification of laboratory chemical products], 6^{th} edition, Elsevier). The ¹H NMR and ¹³C NMR were recorded at 300 or 500 and 75 or 125 MHz respectively, using CDCl₃ (and TMS as internal standard). The δ values are given in parts per million (ppm), the coupling constants (J values) are given in Hertz (Hz), and the multiplicity of the signals is denoted as follows: s, singlet, d, doublet, t, triplet, q, quadruplet, m, multiplet; brs, broad singlet. The high-resolution mass spectrometry (HRMS) analyses were obtained using a Waters Q-TOF 2 apparatus or a Micromass TOF ZABSpec or a micro-TOF Bruker QII or an Orbitrap LTQ XL for electrospray ionization (ESI). The optical rotations were recorded on a Perkin Elmer model 341 polarimeter. The thin layer chromatography was carried out using a precoated silica gel plate (0.2 mm thick).

### 1) Synthesis of the Intermediates of Formula B from the Amino-Ynone of Formula A, Wherein R1 and R2 are as Defined in Formula (I)

### General Procedure for the Heterocyclization Reaction

A mixture of PPh₃AuCl (5 mol %) and AgSbF₆ (5 mol %) is added to the amino-ynone of formula A (0.1 mmol, 1 eq) in 1,2-dichloroethane (1 ml) at ambient temperature under an argon atmosphere. After the resulting mixture has been stirred at ambient temperature for 1 h, Et₂O is added and the resulting mixture is filtered through Celite®. After elimination of the solvents under vacuum, the crude product is purified by silica gel column chromatography using dichloromethane as eluent, to give pure products.

### General Procedure for the Deprotection

The protected dihydropyridone (1 mmol) is dissolved in 5 ml of anhydrous DMF at ambient temperature under the inert atmosphere. The reaction medium is heated at 130° C. for 48 hours. After cooling to ambient temperature, the reaction medium is diluted with 30 ml of water. The product is extracted with dichloromethane (3×20 ml) and the organic phase is washed with a saturated sodium chloride solution and then dried over sodium sulfate. The organic phase is concentrated under reduced pressure and purified by silica gel column chromatography using dichloromethane as eluent.

### 2) Synthesis of the Intermediates of Formula C from the Amino-Ynone of Formula a, Wherein R1 and R2 are as Defined in Formula (I)

### General Procedure for the Tandem Heterocyclization/Halogenation Reaction:

NXS (1.5 eq) is added to the amino-ynone of formula A (0.1 mmol, 1 eq) in 1,2-dichloroethane (1 ml) at ambient temperature under an argon atmosphere. After 5 minutes, a mixture of PPh₃AuCl (5 mol %) and AgSbF₆ (5 mol %) is added to the solution. The resulting mixture is stirred at ambient temperature for 1 h, then Et₂O is added and the resulting mixture is filtered through Celite®. A saturated aqueous sodium thiosulfate solution is added. After separating by settling out, the organic phase is recovered, washed with a saturated aqueous sodium chloride solution and dried over sodium sulfate. After elimination of the solvents under vacuum, the crude product is purified by silica gel column chromatography using dichloromethane as eluent.

### 3) Analyses of the Intermediates of Formula C

With R1=H, R2=unsubstituted phenyl and X=I, the intermediate of formula C tert-butyl 5-iodo-4-oxo-6-phenyl-3,4-dihydropyridine-1(2H)-carboxylate, having the following profile, is obtained:

Yellow solid, yield: 78%; ¹H NMR (500 MHz, CDCl₃): δ=1.05 (s, 9H), 2.87-2.91 (m, 2H), 4.22-4.27 (m, 2H), 7.38-7.44 (m, 5H); ¹³C NMR (125 MHz, CDCl₃): δ=189.5, 158.7, 151.9, 140.0, 129.7, 128.8, 127.9, 89.7, 83.3, 46.4, 37.7, 27.4; HRMS (ESI, m/z): molar mass calculated for C₁₆H₁₈1 NO₃Na: 422.0229, measured [M+Na]⁺: 422.0228.
With R1=benzyl, R2=n-propyl and X=I, an intermediate of formula C (S)-tert-butyl-2-benzyl-5-iodo-4-oxo-6-propyl-3,4-dihydropyridine-1(2H)-carboxylate, having the following profile, is obtained:

Yellow solid, yield: 87%; [α]_{D}²⁵=+223.6 (c 0.8, CH₂Cl₂); ¹H NMR (500 MHz, CDCl₃): δ=1.02 (t, J=7.4 Hz, 3H), 1.63-1.70 (m, 1H), 2.62 (d, J=17.1 Hz, 1H), 2.78-2.96 (m, 4H), 3.17-3.23 (m, 1H), 4.76-4.80 (m, 1H), 7.11-7.31 (m, 5H); ¹³C NMR (125 MHz, CDCl₃): δ=187.4, 160.2, 151.5, 136.8, 129.4, 128.6, 126.8, 90.6, 83.5, 57.4, 42.2, 38.9, 36.3, 27.9, 21.2, 14.0; HRMS (ESI, m/z): molar mass calculated for C₂₀H₂₆1 NO₃Na: 478.0855, measured [M+Na]⁺: 478.0857.
With R1=—CH2-O-benzyl, R2=n-propyl and X=I, an intermediate of formula C (R)-tert-butyl 2-(benzyloxymethyl)-5-iodo-4-oxo-6-propyl-3,4-dihydropyridine-1(2H)-carboxylate, having the following profile, is obtained:

Orange solid, yield: 87%; [α]_{D}²⁵=+201.9 (c 1.2, CH₂Cl₂); ¹H NMR (300 MHz, CDCl₃): δ=0.98 (td, J=7.2 Hz, J=1.6 Hz, 3H), 1.52 (1, 9H), 1.52-1.60 (m, 2H), 2.81-2.97 (m, 3H), 3.16-3.22 (m, 1H), 3.43-3.49 (m, 1H), 3.58-3.61 (m, 1H), 4.47 (d, syst.AB, J=10.6 Hz, 1H), 4.49 (d, syst.AB, J=10.6 Hz, 1H), 4.84-4.89 (m, 1H), 7.26-7.37 (m, 5H); ¹³C NMR (75 MHz, CDCl₃): δ=187.2, 160.6, 151.8, 137.6, 128.4, 127.7, 127.5, 91.6, 83.6, 73.2, 68.0, 54.8, 42.3, 37.7, 28.0, 21.1, 14.0; HRMS (ESI, m/z): molar mass calculated for C₂₁H₂₈INO₄Na: 508.0961, measured [M+Na]⁺: 508.0963.

### 4) Synthesis of the Compounds D of Formula (I) (R3=Vinylcarboxyethyl)

### Preparation of (E)-tert-butyl 5-(3-ethoxy-3-oxoprop-1-enyl)-4-oxo-6-phenyl-3,4-dihydropyridine-1-(2H)-carboxylate by Heck coupling reaction

Ethyl acrylate (53 μl, 0.5 mmol) and triethylamine (70 μl, 0.5 mmol) are added to a solution of 100 mg (0.25 mmol) of tert-butyl 5-iodo-4-oxo-6-phenyl-3,4-dihydropyridine-1-(2H)-carboxylate in 2 ml of anhydrous DMF. The resulting solution is degassed with argon for 15 min. 28.9 mg (0.05 mmol) of Pd(PPh₃)₄ are added and the mixture is stirred at 110° C. for 24 h, then cooled to ambient temperature. Et₂O is added and the resulting mixture is rinsed with a mixture of Et₂O/CH₂Cl₂ (50/50) and filtered through Celite®. The organic phase is concentrated under reduced pressure. The residue is purified by silica gel column chromatography using a mixture of dichloromethane/diethyl ether, to give the pure product in the form of a yellow oil (140 mg, 0.42 mmol, 78%).

Rf=0.50 (petroleum ether/diethyl ether=50/50).

¹H NMR (300 MHz, CDCl₃): δ=1.05 (s, 9H), 1.23 (t, J=6.1 Hz, 3H), 2.71-2.76 (m, 2H), 4.13 (q, J=6.1 Hz, 2H), 4.21-4.25 (m, 2H), 7.00 (s, 2H), 7.28-7.45 (m, 5H).

¹³C NMR (75 MHz, CDCl₃): δ=193.6, 168.1, 159.2, 152.5, 137.5, 136.2, 130.3, 129.2, 128.4, 120.7, 116.5, 83.3, 60.0, 46.1, 39.0, 27.3, 14.2;

HRMS (ESI, m/z): molar mass calculated for C₂₁H₂₅NO₅Na: 394.1630, measured [M+Na]⁺: 394.1630.

### Preparation of (S,E)-ethyl 3-(6-benzyl-4-oxo-2-propyl-1,4,5,6-tetrahydropyridin-3-yl)acrylate by Heck coupling reaction

Ethyl acrylate (117 μl, 1.1 mmol) and triethylamine (153 μl, 1.1 mmol) are added to a solution of 250 mg (0.55 mmol) of (S)-tert-butyl-2-benzyl-5-iodo-4-oxo-6-propyl-3,4-dihydropyridine-1(2H)-carboxylate in 5 ml of anhydrous DMF. The resulting solution is degassed with argon for 15 min. 64 mg (0.05 mmol) of Pd(PPh₃)₄ are added and the mixture is stirred at 130° C. for 48 h, then cooled to ambient temperature. Et₂O is added and the resulting mixture is rinsed with Et₂O/CH₂Cl₂ (50/50) and then filtered through Celite®. The organic phase is concentrated under reduced pressure. The residue is purified by silica gel column chromatography using a mixture of dichloromethane/diethyl ether, to give the pure product in the form of a yellow oil (140 mg, 0.42 mmol, 78%).

Rf=0.30 (dichloromethane/diethyl ether=80/20).

[α]_{D}²⁵=+96.4 (c 0.96, CH₂Cl₂).

¹H NMR (500 MHz, CDCl₃): δ=0.95 (t, J=7.3 Hz, 3H), 1.27 (t, J=7.1 Hz, 3H), 1.55-1.62 (m, 2H), 2.39-2.50 (m, 3H), 2.59 (dd, J=15.9 Hz, J=4.9 Hz, 1H), 2.84-2.95 (m, 2H), 3.85-3.88 (m, 1H), 4.19 (q, J=7.1 Hz, 2H), 5.69 (bs, 1H), 6.98 (d, J=15.4 Hz, 1H), 7.17 (d, J=7.0 Hz, 2H), 7.27-7.36 (m, 3H), 7.40 (d, J=15.4 Hz, 1H);

¹³C NMR (125 MHz, CDCl₃): δ=190.1, 169.8, 167.2, 137.2, 136.1, 129.0, 127.3, 113.4, 104.6, 59.6, 52.9, 42.1, 40.0, 35.2, 21.6, 14.4, 13.7;

HRMS (ESI, m/z): molar mass calculated for C₂₀H₂₅NO₃Na: 350.1732, measured [M+Na]⁺: 350.1736.

### Preparation of (R,E)-ethyl 3-(6-(benzyloxymethyl)-4-oxo-2-propyl-1,4,5,6-tetrahydropyridin-3-yl)acrylate by Heck coupling reaction

Ethyl acrylate (117 μl, 1.1 mmol) and triethylamine (153 μl, 1.1 mmol) are added to a solution of 121.3 mg (0.25 mmol) of (R)-tert-butyl 2-(benzyloxymethyl)-5-iodo-4-oxo-6-propyl-3,4-dihydropyridine-1(2H)-carboxylate in 5 ml of anhydrous DMF. The resulting solution is degassed with argon for 15 min. 64 mg (0.05 mmol) of Pd(PPh₃)₄ are added and the mixture is stirred at 110° C. for 24 h, then cooled to ambient temperature. Et₂O is added and the resulting mixture was rinsed with Et₂O/CH₂Cl₂ (50/50) then filtered through Celite®. The organic phase is concentrated under reduced pressure. The residue is purified by silica gel column chromatography using a mixture of dichloromethane/diethyl ether, to give the pure product in the form of a yellow oil (140 mg, 0.42 mmol, 78%).

Rf=0.50 (dichloromethane/diethyl ether=70/30).

[α]_{D}²⁵=+17.6 (c 0.98, CH₂Cl₂).

¹H NMR (500 MHz, CDCl₃): δ=1.01 (t, J=7.3 Hz, 3H), 1.28 (t, J=7.1 Hz, 3H), 1.59-1.69 (m, 4H), 2.30-2.57 (m, 4H), 3.47-3.62 (m, 2H), 3.85-3.94 (m, 1H), 4.18 (q, J=7.1 Hz, 2H), 5.82 (bs, 1H), 6.97 (d, J=15.3 Hz, 1H), 7.27-7.35 (m, 5H), 7.37 (d, J=15.3 Hz, 1H);

¹³C NMR (125 MHz, CDCl₃): δ=189.3, 169.8, 167.3, 137.1, 128.7, 128.3, 127.9, 113.7, 104.6, 73.5, 71.2, 59.7, 51.4, 38.6, 35.4, 21.7, 14.4, 13.8; HRMS (ESI, m/z): molar mass calculated for C₂₁H₂₇NO₄Na: 380.1838, measured [M+Na]⁺: 380.1839.

### 5) Synthesis of the Compounds E of Formula (I) (R3=Aminocarboxyalkyl)

### Preparation of tert-butyl 1-methyl-4-oxo-1,4,5,6-tetrahydropyridin-3-ylcarbamate by Buchwald coupling reaction

5-Iodo-1-methyl-2,3-dihydropyridin-4(1H)-one (1 eq) is dissolved in an anhydrous toluene solution under an inert atmosphere at ambient temperature. tert-Butyl carbamate (1.1 eq), potassium tert-butoxide (1.5 eq), copper iodide (1 eq) and N,N′-dimethylethylenediamine (2 eq) are consecutively added. The reaction medium is stirred at 90° C. for 12 hours. After cooling to ambient temperature, the reaction medium is filtered through Celite®. The organic phase is concentrated under reduced pressure and the product is purified by silica gel chromatography.

¹H NMR (500 MHz, CDCl₃): δ=1.47 (s, 9H), 2.53-2.56 (m, 2H), 3.04 (s, 3H), 3.35-3.38 (m, 2H), 6.42 (brs, 1H), 7.80 (s, 1H).

¹³C NMR (125 MHz, CDCl₃): δ=183.5, 154.1, 147.3, 111.1, 79.7, 48.8, 43.5, 34.6, 28.3.

### Example 2

### Measurement of the UV Spectrum and of the SPF of Compounds of Formula (I)

### 1) Protocols

UV Spectrum Measurement:

The products are diluted in CHCl₃ so as to obtain a solution at 10⁻⁴M. The UV spectra are measured using a Uvikon 931 UV-vis spectrophotometer, Serlabo Technologies, Entraigues sur la Sorgue, France. This UV spectrum makes it possible to determine the molar extinction coefficient (mol⁻¹ L cm⁻¹) at the λmax by virtue of the Beer-Lambert law: c=A/l×C;

A=value of the absorbance at the λmax; l=length of the cuvette in cm (l=1);

C=concentration of the solution=1×10⁻⁴ mol/l. The theoretical λmax was determined for certain products by DFT calculation.

FIGS. 1 to 3 show the spectra obtained; the compounds are indicated in these figures by their number which appears in the table below (cf. part 2)).

### SPF Measurement:

a) Protocol I (Rennes):

The SPF measurement is carried out according to the following protocol described in Le Dévéhat F., Malargé A., Couteau C., Coiffard L., Legouin B. “Evaluation of a new UV-filter screening method”, 4^{th} symposium of the AFERP “Biodiversity, chimie des substances naturelles et medicaments” [“Biodiversity, chemistry of natural substances and medicaments”] and 3^{rd} Franco-Pakistani symposium, Besançon (France), Jul. 16-17-18, 2010. A solution of each product dissolved in dimethyl sulfoxide is incorporated at 10% into a simplified emulsion of oil-in-water (O/W) type consisting of liquid paraffin, water and SDS at 10%, the whole mixture taken up in absolute ethanol. The UV spectrum of each solution is recorded between 200 nm and 400 nm in a 10 mm quartz cell on a Uvikon 931 UV-vis spectrophotometer and measured in comparison with a control 10% emulsion solution.

b) Protocol II (Nantes):

The SPF measurement is carried out according to the protocol described in Lohézic-Le Dévéhat F., Legouin B., Couteau C., Boustie J., Coiffard L. “Lichenic extracts and metabolites as UV filters” Journal of Photochemistry and Photobiology B: Biology 120 (2013) 17-28:

An emulsion having the following formulation is used:

| | |
|---|---|
| Ingredients | % (m/m) |

Lipophilic phase

| | |
|---|---|
| Liquid paraffin | 17.00 |
| Cetiol HE ® (PEG-7 glyceryl cocoate) | 5.00 |
| Stearic acid | 5.00 |
| Butylated hydroxytoluene | 0.01 |
| Eumulgin ® B1 (Ceteareth-12) | 1.50 |
| Eumulgin ® B2 (Ceteareth-20) | 1.50 |

Hydrophilic phase

| | |
|---|---|
| Glycerol | 4.00 |
| Rhodicare ® T (Xanthan gum) | 0.90 |
| Sodium propylparaben | 0.05 |
| Sodium methylparaben | 0.05 |
| Triethylamine | 0.30 |
| QS | 100.00 |

The two phases are prepared separately. The hydrophilic phase is then added, with stirring, to the lipophilic phase. The products are finally incorporated at 1% into this O/W emulsion. 30 mg of the whole mixture are distributed over the entire surface area (25 cm²) of a PMMA plate using a finger store. After spreading, 15 mg remain on the finger store. Three plates are prepared per product and nine measurements are carried out. The transmission measurements between 290 and 400 nm for the SPF and between 320 and 400 nm for the UVA-PF are carried out using a spectrophotometer equipped with an integrating sphere (UV Transmittance analyzer UV1000S, Labsphere, North Sutton, US).

The SPF and UVA-PF values are, for the two protocols, calculated from the following equations:

$$SPF = \frac{{\sum }_{290}^{400} {E}_{λ} {S}_{λ} {Δ}_{λ}}{{\sum }_{290}^{400} {E}_{λ} {S}_{λ} {T}_{λ} {Δ}_{λ}}$$

$$UVA - PF = \frac{{\sum }_{315}^{400} {E}_{λ} {S}_{λ} {Δ}_{λ}}{{\sum }_{315}^{400} {E}_{λ} {S}_{λ} {T}_{λ} {Δ}_{λ}}$$

where E_{λ} is the erythemogenic effect of the radiation at the wavelength λ; S_{λ} is the spectral solar irradiance at the wavelength λ and T_{λ} is the spectral transmittance of the product at the wavelength λ, λ_{c} is the critical wavelength which corresponds to the wavelength at which 90% of the area under the curve (AUC) is integrated between 290 and 400 nm. The UVA/UVB ratio corresponds more specifically to the ratio of the area under the curve between 315 and 400 nm to the area under the curve between 280 and 315 nm.

### 2) Results

The compounds of formula (I) according to the invention were tested, along with commercial screening agents (at the end of the table, in bold). The results are the following:

| | | | | | | Colipa | |
|---|---|---|---|---|---|---|---|
| | | | | | | UVA-PF | |
| | | λmax exp | | SPF | | p.I, at | λc |
| | | (theore- | | p.I, at 10% | | 10% | p.I |
| | Structure | tical) | | (p.II, at | UVA/ | (p.II, at | (p.II) |
| | name | nm | ε | 1%) | UVB | 1%) | nm |
| 1 | 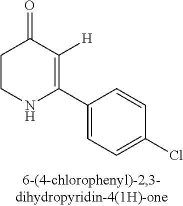 [SMILES: O=C1C=C(c2ccc(Cl)cc2)NCC1] | 328 (305) | 8131 | 2.27 ± 0.06 | 4.78 ± 0.57 | 3.97 ± 0.15 | 366 |
| 2 | 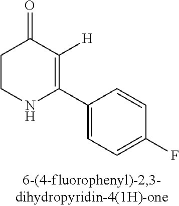 [SMILES: O=C1C=C(c2ccc(F)cc2)NCC1] | 326 (312) | 10924 | 4.30 ± 0.12 (1.81 ± 0.11) | 3.08 ± 0.00 | 5.85 ± 0.16 (1.95 ± 0.12) | 361 (383) |
| 3 | 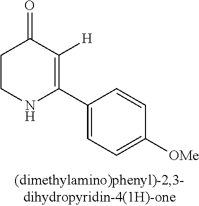 [SMILES: COc1ccc(C2=CC(=O)CCN2)cc1] | 330 (315) | 16259 | 4.30 ± 0.12 (1.25 ± 0.04) | 2.85 ± 0.01 | 9.16 ± 0.10 (1.33 ± 0.04) | 362 (384) |
| 4 | 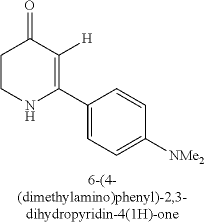 [SMILES: CN(C)c1ccc(C2=CC(=O)CCN2)cc1] | 355 (314) | 26286 | 9.24 ± 0.18 (1.72 ± 0.08) | 5.19 ± 0.01 | 42.67 ± 1.16 (1.88 ± 0.09) | 361 (387) |
| 5 | 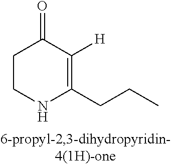 | 303 | 12361 | 12.17 ± 0.09 (11.18 ± 1.03) | 0.36 ± 0.00 | 1.66 ± 0.003 (2.58 ± 0.07) | 328 (335) |
| 6 | 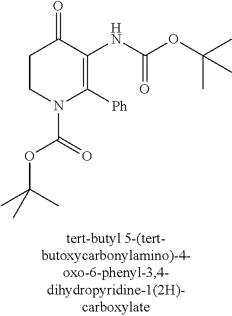 [SMILES: CC(C)(C)OC(=O)NC1=C(c2ccccc2)N(C(=O)OC(C)(C)C)CCC1=O] | 322 | 11206 | 2.04 ± 0.09 | 1.24 ± 0.0 | 1.56 ± 0.04 | 351 |
| 7 | 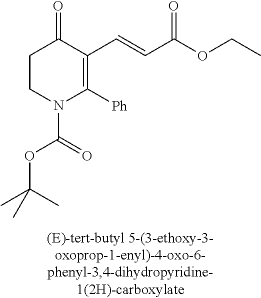 [SMILES: CCOC(=O)/C=C/C1=C(c2ccccc2)N(C(=O)OC(C)(C)C)CCC1=O] | 343 | 15426 | 1.61 ± 0.13 | 6.36 ± 1.16 | 2.92 ± 0.17 | 375 |
| 8 | 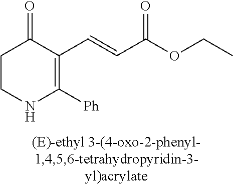 [SMILES: CCOC(=O)/C=C/C1=C(c2ccccc2)NCCC1=O] | 306 | 16596 | 6.73 ± 0.27 | 2.22 ± 0.01 | 5.38 ± 0.16 | 378 |
| 9 | 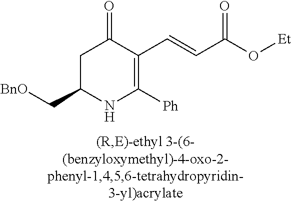 [SMILES: CCOC(=O)/C=C/C1=C(c2ccccc2)N[C@@H](COCc2ccccc2)CC1=O] | 308 | 31339 | 5.40 ± 0.43 | 1.84 | 3.54 | 353 |
| 10 | 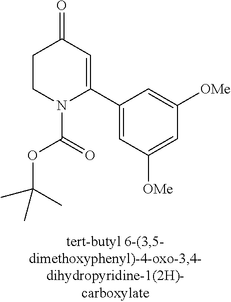 [SMILES: COc1cc(OC)cc(C2=CC(=O)CCN2C(=O)OC(C)(C)C)c1] | 310 | 18622 | 5.54 ± 0.66 | 0.49 ± 0.15 | 1.88 ± 0.01 | 334 |
| 11 | 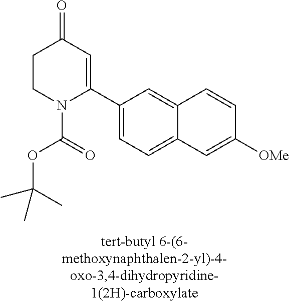 [SMILES: COc1ccc2cc(C3=CC(=O)CCN3C(=O)OC(C)(C)C)ccc2c1] | 331 | 14280 | 5.50 ± 0.62 | 1.92 ± 0.01 | 4.70 ± 0.42 | 362 |
| 12 | 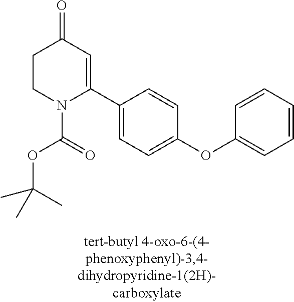 [SMILES: CC(C)(C)OC(=O)N1CCC(=O)C=C1c1ccc(Oc2ccccc2)cc1] | 316 | 20575 | 5.31 ± 0.45 | 0.71 ± 0.01 | 1.98 ± 0.05 | 340 |
| 13 | 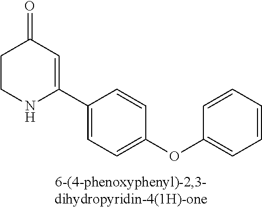 [SMILES: O=C1C=C(c2ccc(Oc3ccccc3)cc2)NCC1] | 333 | 20335 | 2.81 ± 0.11 | 2.99 ± 0.01 | 4.23 ± 0.22 | 360 |
| 14 | 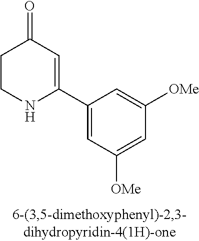 [SMILES: COc1cc(OC)cc(C2=CC(=O)CCN2)c1] | 323 | 12258 | 2.57 ± 1.31 | 2.55 ± 0.85 | 2.86 ± 1.46 | 357 |
| 15 | 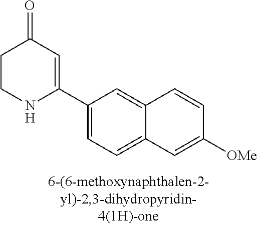 [SMILES: COc1ccc2cc(C3=CC(=O)CCN3)ccc2c1] | 337 | 15578 | 5.89 ± 0.99 | 2.65 ± 0.01 | 7.41 ± 1.34 | 368 |
| 16 | 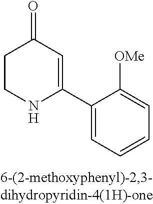 [SMILES: COc1ccccc1C1=CC(=O)CCN1] | 333 | 13110 | 5.94 ± 0.34 | 2.12 | 4.96 ± 0.18 | 358 |
| 17 | 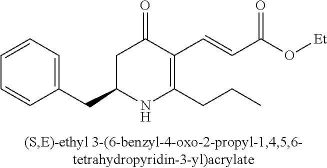 [SMILES: CCCC1=C(/C=C/C(=O)OCC)C(=O)C[C@H](Cc2ccccc2)N1] | 316 | 20260 | 13.28 ± 0.66 | 1.46 ± 0.01 | 4.88 ± 0.11 | 353 |
| 18 | 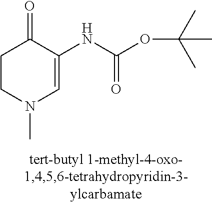 [SMILES: CN1C=C(NC(=O)OC(C)(C)C)C(=O)CC1] | 355 | 8618 | 2.67 ± 0.08 | 3.05 ± 0.04 | 3.49 ± 0.17 | 368 |

| | | | Colipa | λc |
|---|---|---|---|---|
| | SPF | | UVA-PF | p.I |
| Commercial screening | p.I, at 10% | UVA/ | p.I, at 10% | (p.II) |
| agents | (p.II, at 1%) | UVB | (p.II, at 1%) | nm |
| Octyl methoxycinnamate | 10.16 ± 0.02 | 0.32 ± 0.00 | 1.63 ± 0.01 | |
| (OMC) | | | | |
| Diethylhexyl Butamido | 10.10 ± 0.02 | 0.12 ± 0.01 | 1.27 ± 0.01 | |
| Triazone (Uvasorb HEB) | | | | |
| Homosalate | 1.91 ± 0.04 | 0.17 ± 0.00 | 1.08 ± 0.00 | 323 ± 1 |
| PEG-25 PABA | 2.04 ± 0.05 | 0.19 ± 0.01 | 1.11 ± 0.01 | 324 ± 1 |
| Oxybenzone | 5.27 ± 0.25 | 0.98 ± 0.00 | 2.63 ± 0.01 | 349 ± 1 |

The 18 compounds of formula (I) have a λmax located between 300 and 400 nm with an ε greater than 10 000 (mol⁻¹ L cm⁻¹) or even, for 6 of them, greater than 20 000 (mol⁻¹ L cm⁻¹). 10 compounds have an SPF value greater than 5, 17 compounds have a Colipa UVA-PF value of at least 1.66+/−0.003, and 13 of them have a UVA-PF value greater than 3 (whereas the UVA-PF of oxybenzone is 2.63; this product can, however, cause contact allergies and is prohibited in topical anti-sun products for children).

With regard to the λc, the 18 compounds of formula (I) have a value greater than or equal to 328 nm, and 11 compounds have a value greater than 360 nm.

The compounds according to the invention therefore have a very good absorption capacity in the UVA range.

1. A compound of formula (I): 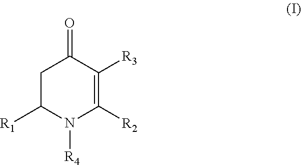
wherein:
R1 is H; benzyl; —CH₂—O-benzyl; a linear or branched C₁-C₁₂ alkyl radical; a hydroxyalkyl radical or a polyhydroxyalkyl radical,
R2 is H; a linear or branched C₁-C₁₂ alkyl radical; a —(CH₂)₂—CH═CH₂ radical; a phenyl, naphthyl, furan, thiophene or pyrazole radical, optionally substituted by at least one group selected from methoxy, phenoxy, halogen, dimethylamine, trifluoromethyl and alkynyl,
R3 is H; a vinyl radical optionally substituted by a carboxyalkyl; a nitrile; a carboxamide; a phenyl, naphthyl, furan, thiophene or pyrazole radical, optionally substituted by at least one group selected from methoxy, phenoxy, halogen, dimethylamine, trifluoromethyl and alkynyl; an aminoalkyl or aminocarboxyl radical; or an alkoxy radical,
R4 is H; a protective group; a linear or branched C₁-C₁₂ alkyl radical; a hydroxyalkyl radical or a polyhydroxyalkyl radical,
it being understood that:
when R2 is H, then R3 is —NHCOOC(CH₃)₃,
when R2 is a propyl, then R3 is —CH═CH—COO—C₂H₅ or R1=R3=R4=H,
when R2 is an unsubstituted phenyl, then R3 is selected from —CH═CH—COO—C₂H₅ and —NHCOOC(CH₃)₃, and
when R2 is a phenyl substituted in the para position, then R3 is H.

2. The compound as claimed in claim 1, wherein the protective group is a group of the carbamate family, preferably a group selected from tert-butoxycarbonyl, carbobenzyloxy and 9-fluorenylmethyloxycarbonyl.

3. The compound as claimed in claim 1, wherein the halogen is selected from iodine, bromine, chlorine and fluorine.

4. The compound as claimed in claim 1, wherein R2 is selected from a propyl radical, a —(CH₂)₃—CH═CH₂ radical, an unsubstituted phenyl, 4-chlorophenyl, 4-fluorophenyl, 2-methoxyphenyl, 4-methoxyphenyl, 4-dimethylaminophenyl, 3,5-dimethoxyphenyl, 3-methoxynaphthyl and 4-phenoxyphenyl.

5. The compound as claimed in claim 1, is selected from:
6-(4-chlorophenyl)-2,3-dihydropyridin-4(1H)-one,
6-(4-fluorophenyl)-2,3-dihydropyridin-4(1H)-one,
6-(4-methoxyphenyl)-2,3-dihydropyridin-4(1H)-one,
6-(4-(dimethylamino)phenyl)-2, 3-dihydropyridin-4 (1H)-one,
6-propyl-2,3-dihydropyridin-4(1H)-one,
tert-butyl 5-(tert-butoxycarbonylamino)-4-oxo-6-phenyl-3,4-dihydropyridine-1(2H)-carboxylate,
(E)-tert-butyl 5-(3-ethoxy-3-oxoprop-1-enyl)-4-oxo-6-phenyl-3,4-dihydropyridine-1(2H)-carboxylate,
(E)-ethyl 3-(4-oxo-2-phenyl-1,4,5,6-tetrahydropyridin-3-yl)acrylate,
(R,E)-ethyl 3-(6-(benzyloxymethyl)-4-oxo-2-phenyl-1,4,5,6-tetrahydropyridin-3-yl) acrylate,
tert-butyl 6-(3,5-dimethoxyphenyl)-4-oxo-3,4-dihydropyridine-1(2H)-carboxylate,
tert-butyl 6-(6-methoxynaphthalen-2-yl)-4-oxo-3,4-dihydropyridine-1(2H)-carboxylate,
tert-butyl 4-oxo-6-(4-phenoxyphenyl)-3,4-dihydropyridine-1(2H)-carboxylate,
6-(3,5-dimethoxyphenyl)-2,3-dihydropyridin-4(1H)-one,
6-(4-phenoxyphenyl)-2,3-dihydropyridin-4(1H)-one,
6-(6-methoxynaphthalen-2-yl)-2,3-dihydropyridin-4(1H)-one,
6-(2-methoxyphenyl)-2,3-dihydropyridin-4(1H)-one,
(S,E)-ethyl 3-(6-benzyl-4-oxo-2-propyl-1,4,5,6-tetrahydropyridin-3-yl)acrylate, and
tert-butyl 1-methyl-4-oxo-1,4,5,6-tetrahydropyridin-3-ylcarbamate.

6. A composition comprising, in a cosmetically acceptable medium, at least one compound of formula (I) as claimed in claim 1.

7. The composition as claimed in claim 6, further comprising at least one additional compound which screens out UVA and/or UVB radiation.

8. The composition as claimed in claim 7, wherein the additional compound which screens out UVA and/or UVB radiation is selected from dibenzoylmethane derivatives, aminobenzophenones, anthranilic derivatives, 4,4-diarylbutadiene derivatives, merocyanine derivatives, bisbenzoazolyl derivatives, para-aminobenzoates, salicylic derivatives, cinnamates, benzylidenecamphor derivatives, triazine derivatives, imidazoline derivatives, benzalmalonate derivatives, phenylbenzimidazolesulfonic acid, ferulic acid, salicylic acid, benzophenone derivatives, phenyl benzotriazole derivatives, benzoxazole derivatives, bisresorcinyltriazine derivatives and mineral screening agents, which are coated or uncoated pigments.

9. The non-therapeutic cosmetic use of at least one compound of formula (I) as claimed in claim 1, for protecting the skin and/or the lips and/or the hair against solar radiation.

10. A cosmetic process for protecting the skin and/or the lips and/or the hair, comprising the application of at least one compound of formula (I) as claimed in claim 1 to the skin and/or the lips and/or the hair.

11. A cosmetic process for protecting the skin and/or the lips and/or the hair, comprising the application of at least a composition as defined in claim 6, to the skin and/or the lips and/or the hair.
